(19) United States
(12) Patent Application Publication
(10) Pub. No.: US 2025/0285718 A1
(43) Pub. Date: Sep. 11, 2025

(54) SYSTEMS AND METHODS FOR AUTOMATIC MEDICAL REPORT GENERATION

(71) Applicant: Shanghai United Imaging Intelligence Co., Ltd., Shanghai (CN)

(72) Inventors: Benjamin Planche, Briarwood, NY (US); Ziyan Wu, Lexington, MA (US); Meng Zheng, Cambridge, MA (US); Zhongpai Gao, Rowley, MA (US); Abhishek Sharma, Boston, MA (US); Terrence Chen, Lexington, MA (US); Xiao Chen, Lexington, MA (US); Lin Zhao, Billerica, MA (US); Xiao Fan, Cambridge, MA (US); Zhang Chen, Brookline, MA (US); Yikang Liu, Cambridge, MA (US); Shanhui Sun, Lexington, MA (US); Arun Innanje, Lexington, MA (US); Wenzhe Cui, Cambridge, MA (US)

(73) Assignee: Shanghai United Imaging Intelligence Co., Ltd., Shanghai (CN)

(21) Appl. No.: 18/599,533

(22) Filed: Mar. 8, 2024

(51) Int. Cl. G16H 15/00; G06N 20/00; G16H 10/60; G16H 30/20

(52) U.S. Cl. CPC G16H 15/00; G06N 20/00; G16H 10/60; G16H 30/20

(57) ABSTRACT

The decision process of a first machine learning (ML) model may be explained based on a second ML model implemented on an apparatus. The apparatus may obtain a prediction about an image made based on the first ML model. The apparatus may further determine visual concepts associated with the image that may have been used by the first ML model to make the prediction, and determine respective contributions of the visual concepts to the prediction made by the first ML model. The apparatus may then generate, based on the second ML model, a textual description that explains the respective contributions of the visual concepts to the prediction made by the first ML model. The second ML model may determine respective image features associated with the visual concepts, map the determined image features to corresponding text features, and generate the textual description based at least on the text features.

### BACKGROUND

Medical reports may provide a detailed account of medical procedures (e.g., such as surgical procedures) performed on a patient. These reports typically contain a standardized set of information, including the patient's medical history, the reason for performing the procedure, the medical techniques used, any complications or unexpected events that occurred during the procedure, and/or postoperative plans for the patient's care. The reports are usually written by a member of the medical team attending to the patient immediately after the procedure, while the details are still fresh in their minds. The format and content of these medical reports may vary depending on the type of medical procedure performed and/or an institution's specific requirements. However, the reports generally follow a structured format to ensure that all relevant information is included and organized in a clear and concise manner.

Manual generation of medical reports can be a time-consuming and error-prone process. Firstly, it may take a significant amount of time for healthcare professionals to create detailed and accurate reports. Furthermore, manual reports may vary in structure and content depending on the individual writing them, leading to inconsistencies and omissions of crucial details. Handwritten reports may also be difficult to read and understand, especially if the handwriting is not legible. In addition, there is a risk of human error when creating manual reports, and mistakes in recording medical details and complications may lead to inaccurate or incomplete records that may negatively impact patient care in the future.

Accordingly, systems and methods that can automate the medical report generation process and help overcome the challenges described above may be desirable.

### SUMMARY

Described herein are systems, methods, and instrumentalities associated with automatic medical report generation. According to embodiments of the present disclosure, an apparatus may obtain at least a first type of data associated with a medical procedure and a second type of data associated with the medical procedure. The apparatus may generate, using a first machine learning (ML) model, first textual descriptions based on the first type of data, wherein the first textual descriptions may be associated with multiple temporal levels. The apparatus may further generate, using a second ML model, second textual descriptions based on the second type of data, wherein the second textual descriptions may be also associated with the multiple temporal levels. The apparatus may then produce a raw medical report that describes the medical procedure based at least on the first textual descriptions and the second textual descriptions, wherein the first textual descriptions and the second textual descriptions may be aggregated in the raw medical report based on the multiple temporal levels with which the first textual descriptions and the second textual descriptions are associated. The apparatus may refine the raw medical report based on a large language model (LLM).

In examples, the first type of data may include a video recording of the medical procedure, and the first ML model may include a vision-language model configured to extract visual features from the video recording and generate the first textual descriptions based on the extracted visual features. In these examples, the second type of data may include an audio recording of the medical procedure, and the second ML model may include a speech recognition model configured to extract sound features from the audio recording and generate the second textual descriptions based on the extracted sound features. Alternatively, or additionally, the second type of data may include patient vital signs, patient medical records, or logs of a device used during the medical procedure, and the second ML model may include an ML model configured to extract features from the patient vital signs, the patient medical records, or the logs of the device used during the medical procedure, and to map the extracted features to the second textual descriptions.

In examples, the vision-language model described herein may determine, for each frame of the video recording, one or more region-wise tokens each indicative of a person or object detected in a corresponding region. For each frame of the video recording, the vision-language model may further determine a caption that describes the frame.

In examples, each of the multiple temporal levels described herein may correspond to a respective time spot or step of the medical procedure. In examples, the apparatus may produce the raw medical report by concatenating, for each temporal level of the multiple temporal levels, one or more of the first textual descriptions that correspond to the temporal level with one or more of the second textual descriptions that correspond to the temporal level, and then aggregating the one or more of the first textual descriptions and the one or more of the second textual descriptions that are concatenated at each temporal level across the multiple temporal levels.

In examples, the LLM described herein may utilize a transformer architecture and may have over one billion parameters. The LLM may be configured to refine the raw medical report based on a predefined report structure or predefined report language.

In examples, the LLM may be pre-trained to detect abnormalities in the raw medical report, wherein refining the raw medical report based on the LLM may include providing an indication of the abnormalities detected in the raw medical report.

In examples, the LLM may be pre-trained to replace a medical terminology included in the raw medical report with descriptive texts, wherein refining the raw medical report based on the LLM may include replacing the medical terminology with the descriptive texts.

In examples, the LLM may be pre-trained to determine, based on the first type of data or the second type of data, standard operations associated with the medical procedure and actual operations being performed in the medical procedure, wherein the apparatus may be further configured to detect inconsistency between the actual operations and the standard operations, and provide an indication of the inconsistency.

### BRIEF DESCRIPTION OF THE DRAWINGS

A more detailed understanding of the examples disclosed herein may be had from the following descriptions, given by way of example in conjunction with the accompanying drawings.

FIG. 1 is a simplified block diagram illustrating an example of automatic report generation according to embodiments of the present disclosure.

FIG. 2A is a simplified block diagram illustrating an example of multi-level textual descriptions based on multi-modal data according to embodiments of the present disclosure.

FIG. 2B is a simplified block diagram illustrating an example of generating and refining a raw medical report according to embodiments of the present disclosure.

FIG. 3 is a simplified block diagram illustrating an example of training a vision-language model according to embodiments of the present disclosure.

FIG. 4 is a simplified block diagram illustrating an example of a neural network architecture that may be used to accomplish vision-language understanding and generation according to embodiments of the present disclosure.

FIG. 5 is a flow diagram illustrating example operations associated with training an artificial neural network to perform one or more of the tasks described in embodiments of the present disclosure.

FIG. 6 is a simplified block diagram illustrating an example apparatus that may be configured to perform one or more of the tasks described in embodiments of the present disclosure.

### DETAILED DESCRIPTION

The present disclosure is illustrated by way of example, and not by way of limitation, in the figures of the accompanying drawings. A detailed description of illustrative embodiments will now be provided with reference to these figures. Although these embodiments may be described with certain technical details, it should be noted that the details are not intended to limit the scope of the disclosure.

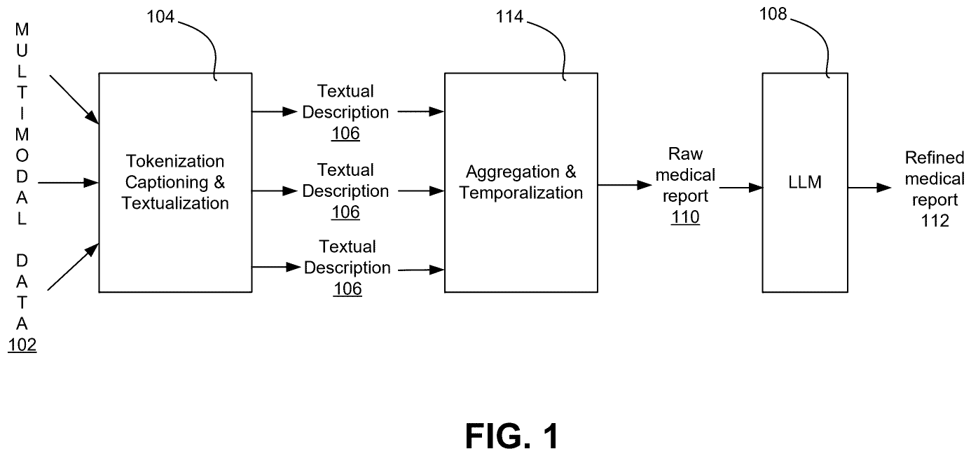

FIG. 1 illustrates an example of automated medical report generation based on one or more machine learning (ML) models. The medical report may be any type of written description of a medical procedure such as, e.g., a surgical report that provides a detailed account of surgical procedures performed on a patient. As shown in FIG. 1, the medical report may be generated based on multi-modal data 102 that may include various modalities or types of data such as, e.g., video recordings, audio recordings, patient vital data (e.g., collected via cardiogram recordings), patient files (e.g., medical history, scan images, etc.), medical device logs, etc. The multi-modal data may be collected using various sensing devices including, for example, visual sensors (e.g., red-green-blue (RGB) sensors, depth (D) sensors, RGB-D sensors, and/or infrared sensors), audio sensors (e.g., any type of mono or stereo microphone embedded with a visual sensor or standing-alone), vita sign monitoring devices (e.g., patient heart rate, patient respiratory rate, patient body temperature, clinician fatigue level, etc.), and/or the like. The various sensing devices may be installed in or about the environment in which the medical procedure is performed, and may be calibrated and parameterized to share a common time referential that may facilitate the temporal alignment of the multimodal data, as will be described in greater detail below. The multi-modal data may also be retrieved from a medical record repository (e.g., patient medical history database) and/or received via one or more textual inputs, such as the logs of any relevant machine used for the medical procedure.

The machine learning models used to generate the medical report may include one or more models pre-trained to tokenize, captioning, and/or textualizing the multi-modal data at 104 to derive respective textual descriptions 106. The machine learning models used to generate the medical report may also include a large language model (LLM) 108 that may be pre-trained to refine a raw medical report 110 generated from the textual descriptions 106 into a final medical report 112. As will be described in greater detail below, textual descriptions 106 may be generated at a fine level first (e.g., per time spot or step of the medical procedure and/or per region across each image frame), and then temporalized and aggregated at 114 to derive the raw medical report 110 (e.g., by combining the descriptions for multiple time spots into a description for a segment, and/or combining the descriptions for multiple regions into a description a frame). The raw medical report may then be refined into the final medical report 112 leveraging LLM 108, which may be configured to iterate over the raw medical report and output a summarized version based on predefined structure and language of medical reports.

The artificial intelligence (AI) based approach illustrated in FIG. 1 may help overcome challenges associated with manual report generation, such as, e.g., time spent on report writing, human errors, inconsistencies, etc. The ML models used in the AI-powered approach may be trained on publicly available data (e.g., from the Internet and/or published medical journals). Once trained and given multi-modal data regarding a medical procedure (e.g., video recordings, audio recordings, patient files, etc.), the ML models may extract relevant information from the various modalities, generate multi-level tokens (e.g., region-wise tokens), captions (e.g., frame-wise captions), and/or other structured information based on the extracted information (e.g., via temporal and/or structural encoding), and compose a medical report that describes the medical procedure utilizing a pre-trained large language model.

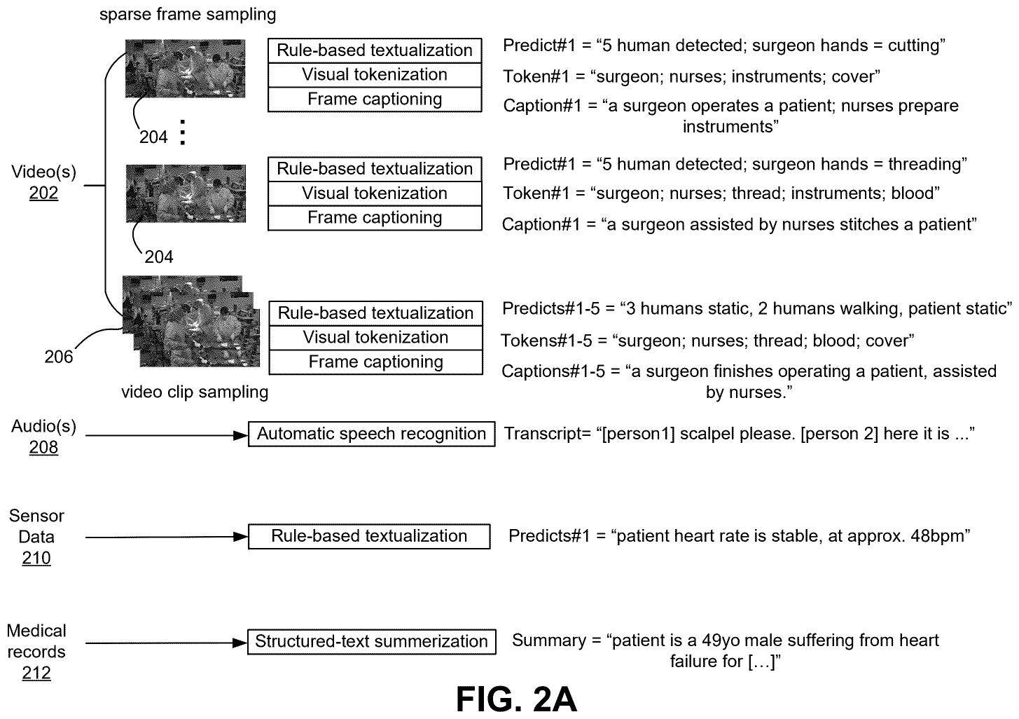

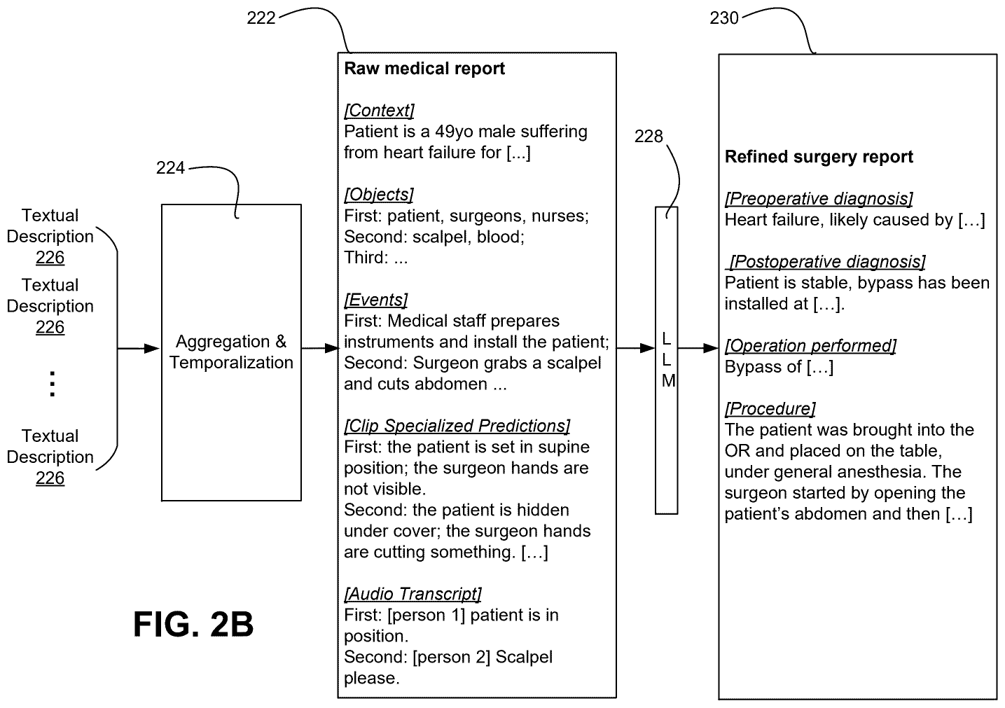

FIG. 2A and FIG. 2B illustrate an example of generating a medical report based on multiple types of data including videos, audios, sensor data (e.g., patient vital signs, device logs, etc.), medical records (e.g., patient medical history), etc. The various types of data may be collected during a medical procedure (e.g., surgery) and, as shown in FIG. 2A, may be converted into textual descriptions in a multi-level (e.g., pyramidal) manner. For example, given one or more video recordings 202 of a medical procedure (e.g., surgery), a machine learning model (e.g., referred to herein as a vision-language model) with both natural language processing (NLP) and computer vision (CV) capabilities may be used to analyze the content of the video, extract meaningful information from the visual content, and generate natural language descriptions of the visual content. For example, the natural language descriptions may include scene/event predictions (e.g., labeled “Predict” in FIG. 2A), tokens (e.g., labeled “Token” in FIG. 1A), and/or frame captions (e.g., labeled “Caption” in FIG. 2A).

The vision-language model may be configured to generate the textual descriptions in a multi-level manner. For instance, the vision-language model may perform sparse frame sampling of each image frame 204 of the video 202 that may correspond to a specific time spot or step of the medical procedure, and generate text that describes the people, objects, and/or events detected by the vision-language model from the image frame. The vision-language model may also perform video clip sampling of video segments 206 of video 202 (e.g., each such segment may include multiple image frames), and generate text that describes the people, objects, and/or events (e.g., including movement of the people and/or objects) observed in the video clip. Each video segment 206 may be processed at once as a unit (e.g., a 3D volume derived based on frame height×frame width×frame number). The textual description for each image frame may correspond to a first temporal (e.g., in terms of the time spot or procedural step associated with the image frame) or structural (e.g., per frame) level, while the textual description for each video clip may correspond to a second temporal (e.g., in terms of the time duration or procedural segment associated with the video clip) or structural (e.g., per video clip) level. In this manner, the approach illustrated by FIG. 2A may be used to generate textual descriptions for a long video recording (e.g., hours-long) in a pyramidal manner, starting from frame-wise data, then combining the results for short clips, before aggregating the descriptions over the whole video. The textual descriptions derived for video recordings 202 at each level (e.g., temporal level) may also be combined with the textual descriptions of other modalities at the same level to generate a more comprehensive report.

The vision-language model may be trained to derive the multi-level textual descriptions from image frames 204 and/or video clip 206 using various rule-based textualization, visual tokenization, and/or frame captioning techniques. For example, the vision-language model may be implemented via a transformer neural network with built-in self-attention and/or cross-attention mechanisms that may be configured to encode the features of image frames 204 and/or video clip 206 into image embeddings, and then decode those embeddings into a description of the people, objects, activities, and/or events captured in the image frames 204 and/or video clip 206. The text conversion capabilities of the vision-language model may be enhanced by other user-provided algorithms. For examples, while a pre-trained vision transformer may be used to predict a human activity depicted in an image frame as “stitching a patient,” an additional rule-based module or model may be used to map the categorical prediction into a natural language sentence such as “a person is stitching the patient.” The vision-language model may additionally be enhanced by domain-specific recognition techniques, such as gesture recognition, tracking, and/or human body modeling, to extract further structured semantic information from the image frames 204 and/or video clip 206 and convert the extract information into text based on predefined rules.

As shown in FIG. 2A, the automatic report generation techniques described herein may account for other (e.g., non-visual) modalities relevant to the medical procedure such as audio recordings 208, sensor data 210, and/or medical records 212 that may be associated with the medical procedure or the patient. Similar to video recordings 202, these other modalities may be converted into textual descriptions using techniques specially designed for each type of data. For example, the audio recordings may be transcribed into text descriptions using automatic speech recognition techniques, the sensor data (e.g., vital signs of the patient) may be converted into a textual description using rule-based textualization techniques, and the medical records may be summarized into a text description using structured-text summarization techniques. In examples, the speech recognition techniques may be implemented via a speech recognition ML model that may be pre-trained to extract sound features from audio recordings 208 and generate the corresponding textual descriptions based on the extracted sound features. In examples, the rule-based textualization techniques may involve using predefined rules and patterns to convert non-textual sensor data, such as numerical values or categorical labels, into human-readable text. For instance, using an example rule-based textualization technique, numerical measurements of a patient's body temperature may be converted into text by applying rules devised based on medical knowledge (e.g., if the temperature is between 37 and 38° C., then the output text may be “patient temperature is normal,” and if the temperature is above 38° C., the output text may be “patient has fever”). The structured-text summarization techniques may involve condensing the medical records that may follow a structured format into a shorter version while retaining the key information and structure. The summarization may also involve generating new sentences to convey the essence of the original content. The summarization may be accomplished utilizing various types of neural networks, such as recurrent neural networks (RNNs) or transformer neural networks, with the latter implementing an attention mechanism to focus on relevant parts of the input text when generating the summary.

Also similar to video recordings 202, one or more of audio recordings 208, sensor data 210, and/or medical records 212 may be processed in a multi-level manner. For instance, similar to video recordings 202, different segments of audio recordings 208, sensor data 210, and/or medical records 212 may also correspond to different time spots or steps of the medical procedure and therefore the audio recordings, sensor data, and/or medical records may also be converted into textual descriptions at a temporal level that corresponds to a respective time spot or step of the medical procedure such that the textual descriptions may subsequently be aggregated within themselves to derive longer sentences or paragraphs, and/or with the textural descriptions of video recordings 202 to derive a more comprehensive report.

FIG. 2B illustrates an example of medical report generation and refinement. As shown in FIG. 2B, a raw medical report 222 of a medical procedure may be generated by aggregating and temporalizing, at 224, textual descriptions 226 that may be predicted based on different types of data (e.g., multi-modal data) associated with the medical procedure. The aggregation and temporalization may be performed using ML-based or non-ML-based techniques. As described herein, textual descriptions 226 may be associated with different temporal and/or structural levels (e.g., frame-wise, segment-wise, etc.) and, as such, the aggregation and temporalization at 224 may involve aggregating the textual descriptions corresponding to smaller temporal units into larger temporal segments and/or across multiple modalities. For example, the aggregation may be accomplished by aggregating the textual descriptions of different temporal levels for each modality separately and then combining the aggregation results for all modalities together. As another example, the aggregation may also be accomplished by first concatenating the textual descriptions from different modalities at the same temporal level and then aggregating them all together across the temporal levels. For instance, the tokens shown in FIG. 2A may be statistically aggregated (e.g., based on their frequency), whereas the captions (e.g., those labeled “Caption”) and structured information (e.g., those labeled “Predict”) may be summarized based on a pretrained summarization model. As an example, if the outputs are textual descriptions generated by language models, the per-frame descriptions may be summarized over the considered video (sub-) segment by another summarization language model. If the outputs are tokens or feature vectors, they may be aggregated via weighted summation. In some examples, in addition to aggregating the text descriptions obtained from the different modalities, temporal (e.g., “first/second/third/etc.” or “at minute 0/1/etc.”) and/or structural (e.g., “objects,” “actions,” “audio,” “history,” etc.) labels or tokens may also be added to the raw medical report 222 to guide subsequent steps of report generation. For instance, the temporal and/or structural tokens may be added to the predictions accordingly to predefined rules (e.g., a “firstly” token may be added to predictions corresponding to the 1st video segment) to provide contextual information to the following summarization models and/or inject known priors and metadata (e.g., temporal ordering of the video segments, nature of the sensors generating the data, etc.) into the intermediary results to provide more context for the downstream summarization.

As shown in FIG. 2B, the report generation process described herein may include passing the raw medical report 222 to a large language model (LLM) 228 such that the raw report may be digested and summarized into a refined report 230. LLM 228 may include a large number of parameters (e.g., billions) and may be trained on enormous amounts of data to understand and generate coherent and contextually relevant text like a human. In examples, LLM 228 may be built on a transformer architecture with built-in attention mechanisms that allow the model to focus on different parts of an input sequence when making predictions, enabling it to capture long-range dependencies in language. For purposes of generating the refined report 230, LLM 228 may be parameterized and finetuned according to a predefined structure and language expected for medical reports (e.g., the LLM may be trained with domain-relevant data such as existing surgery video recordings and corresponding reports).

In examples, LLM 228 may be configured to replace descriptions in the raw medical report 222 that may be phrased in professional terms with language that a layperson or common person without expertise on the subject can understand. LLM 228 may also summarize the key findings of raw medical report 222 and highlight important information in the report for a reader's attention. Additionally, LLM 228 may be trained for generative and/or interactive report composition. For example, based on knowledge acquired from past clinical diagnoses and analyses, and upon encountering a certain word or phrase in the raw report 222, LLM 228 may generate additional words, sentences, or paragraphs that are commonly seen with the encountered word or phrase in the relevant application setting.

In examples, LLM 228 may be configured to identify discrepancies and/or contradictions in the raw medical report 222, and correct those discrepancies and/or contradictions based on knowledge that LLM 228 may acquire through training. In these examples, LLM 228 may include or may be used in conjunction with one or more of a pre-processing module, a validation module, a feedback module, or a user interface module. The pre-processing module may be responsible for preparing the raw medical report 222 for input into LLM 228, such as, e.g., by removing irrelevant information from the report and ensuring that the report is in a format that can be used by LLM 228. The validation module may be responsible for validating the findings of LLM 228 (e.g., regarding discrepancies and/or contradictions), for example, by comparing the findings with existing reports and standards to determine if they are indeed errors or inconsistencies. A confidence score may be provided to indicate the accuracy of the detected errors or inconsistencies. The feedback module may be responsible for providing feedback to LLM 228 to improve the model's understanding of medical terminologies and relationships between words and phrases. The user interface module may be responsible for allowing a user to interact with LLM 228, such as, e.g., reviewing and approving the report generated by LLM 228.

In examples, in addition to refining raw medical report 222, LLM 228 may be further trained to determine, based on the multi-modal data described herein, standard operations (e.g., for quality assurance purposes) of the medical procedure and actual operations that may be performed in the medical procedure. LLM 228 may then compare the actual operations with the predicted standard operations (e.g., in real time), and provide an indication of any inconsistency or discrepancy detected from the comparison (e.g., the inconsistency may indicate that the medical procedure is not being performed following quality assurance guidelines). LLM 228 may be trained to acquire domain knowledge about the medical procedure based on publicly available records, documents, videos, audios, etc. regarding the medical procedure. In examples, LLM 228 may be further trained to accept natural language text and/or image embeddings as inputs, and generate instructions or guidance about the medical procedure that may consider the context of the medical procedure (e.g., medical conditions of the patient). For example, LLM 228 may be trained to recognize an individual's acts or speech based on video, audio and/or text embeddings, interpret the individual's intention based on the acts or speech as well as the surrounding context, and provide a response accordingly. As another example, LLM 228 may infer, based on the relationships between people and/or objects observed in a scene, the stage that a medical procedure is in and/or the activities being performed. The model may then compare the stage and/or activities with the model's internal knowledge about the medical procedure, and determine whether the medical procedure is following proper protocols.

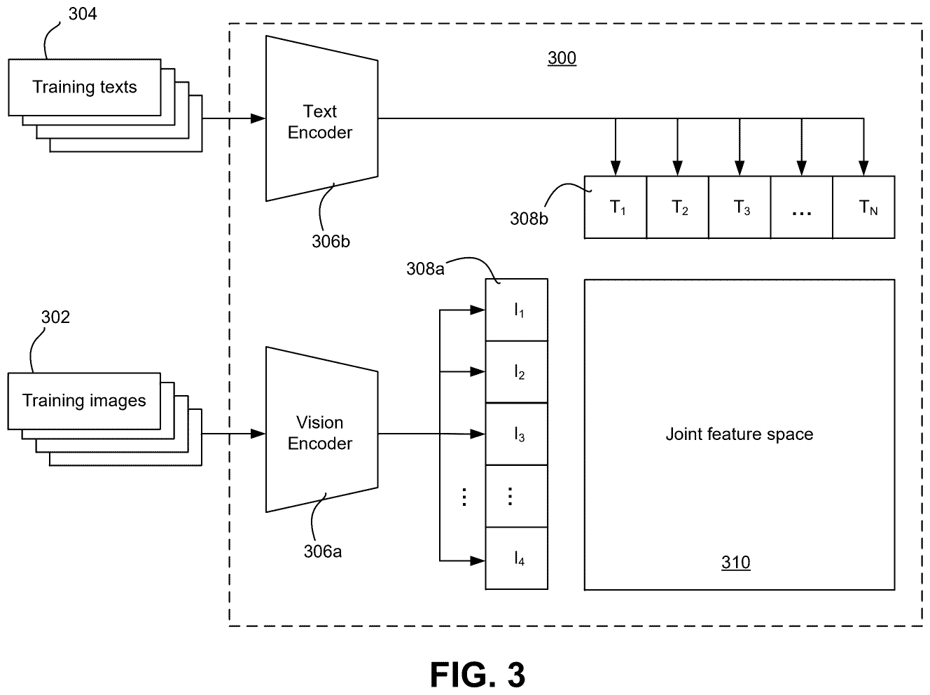

FIG. 3 illustrates an example of training a vision-language model 300 to learn a mapping between visual and textual embeddings (e.g., between visual and textual features) such that, when given an image, the vision-language model may generate descriptive text based on features extracted from the image. As shown in FIG. 3, the vision-language model may be trained using a dataset comprising paired images 302 and textual descriptions 304. The training dataset may be obtained from various sources including, for example, the Internet (e.g., websites that may include images and descriptions of the images), publicly accessible databases (e.g., figures and captions from academic publications), hospital records (e.g., radiology reports), etc. The training data may be pre-processed, for example, to ensure that it is in a suitable format for the training. The pre-processing may, for example, include resizing the images, tokenizing the text, creating pairs of image-text inputs, etc. The pre-processing may also include augmenting the training data (e.g., by varying the textual descriptions to increase the diversity of the training dataset) to improve the robustness and accuracy of the vision-language model 300.

The vision-language model 300 may include a vision encoding portion (e.g., implemented via a vision encoder 306a) and a text encoding portion (e.g., implemented via a text encoder 306b). In examples, the vision encoder 306a may utilize a vision transformer architecture designed to extract image features 308a from input images 302, while the text encoder 306b may be implemented using a regular transformer architecture designed to extract text features 308b from textual descriptions 304. The image features 308a and text features 308 may then be aligned (e.g., mapped to each other) in a joint embedding space 310 (e.g., through concatenation or some other suitable fusion techniques) to capture the relationships between the visual and textual information. In examples, the vision encoder 306a and the text encoder 306b may be trained first (e.g., separately) on a large number of images and textual descriptions, respectively, and then fine-tuned using an application specific dataset (e.g., images from surgical videos) and/or based on a specific downstream task (e.g., medical report generation).

In examples, a contrastive learning technique may be employed to force the vision-language model 300 to bring similar image-text pairs closer in the joint embedding space 310, while pushing dissimilar image-text pairs further apart. Various contrastive loss functions may be used for this purpose including, for example, those based on normalized temperature-scaled cross-entropy (NT-Xent) or information noise-contrastive estimation (InfoNCE). The contrastive learning may help the vision-language model 300 acquire an understanding of the relationships between certain visual and textual embeddings or features such that, when given an image (e.g., the image frames or video clips described herein) as inputs, vision-language model 300 may extract visual features from those inputs and generate a coherent and informative explanation of the visual content contained in the inputs. Vision-language model 300 may do so, for example, by relating the extracted visual features to corresponding textual features (e.g., textual descriptions) in the learned joint embedding space 310.

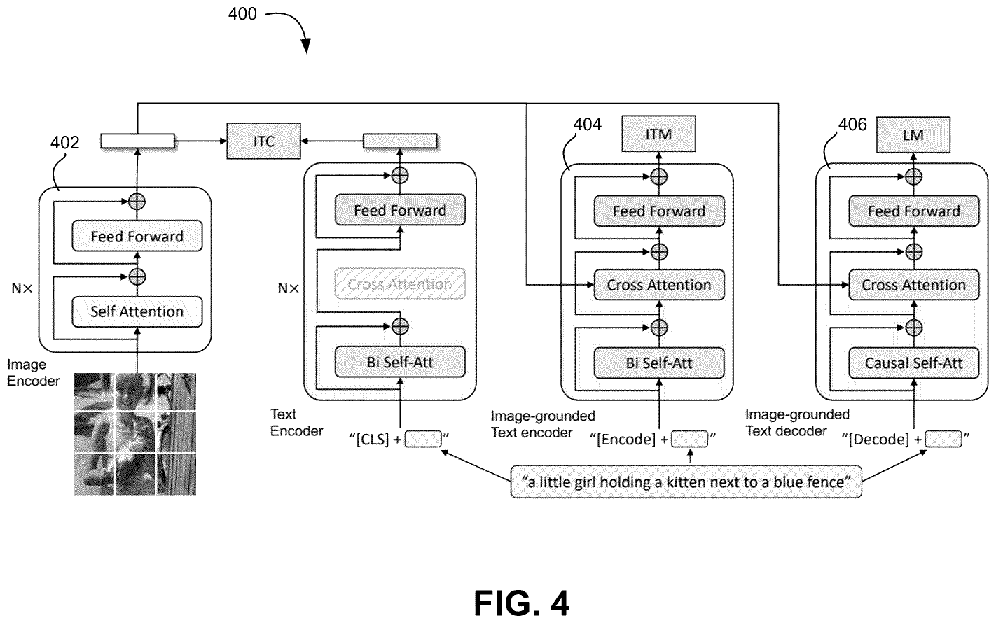

FIG. 4 illustrates an example of a neural network architecture 400 that may be used to accomplish unified vision-language understanding and generation. As shown, neural network architecture 400 may include a unimodal encoder 402, an image-grounded text encoder 404, and an image-grounded text decoder 406. The unimodal encoder 402 may be configured to separately encode images and texts and may be trained with an image-text contrastive (ITC) loss to align vision and language representations. The image-grounded text encoder 404 may be configured to model vision-language interactions using one or more cross-attention layers, and may be trained with an image-text matching (ITM) loss to distinguish between positive and negative image-text pairs. The cross-attention layers may be inserted between a self-attention layer and a feed forward for each transformer block of the text encoder 404. The image-grounded text decoder 406 may be trained with a language modeling (LM) loss to generate a frame caption when given an image. Decoder 406 may replace one or more bi-directional self-attention layers that may exist in encoder 404 with causal self-attention layers, while sharing the same cross-attention layers and feed forward networks as the encoder. Using a multimodal mixture of encoder-decoder, neural network architecture 400 may achieve effective multi-task pre-training and flexible transfer learning. The neural network may be jointly pre-trained using noisy image-text pairs and with three objectives: image-text contrastive learning, image-text matching, and image-conditioned language modeling. The neural network may then be finetuned into two modules: a captioner to produce synthetic captions given images, and a filter to remove noisy captions from both original texts and synthetic texts.

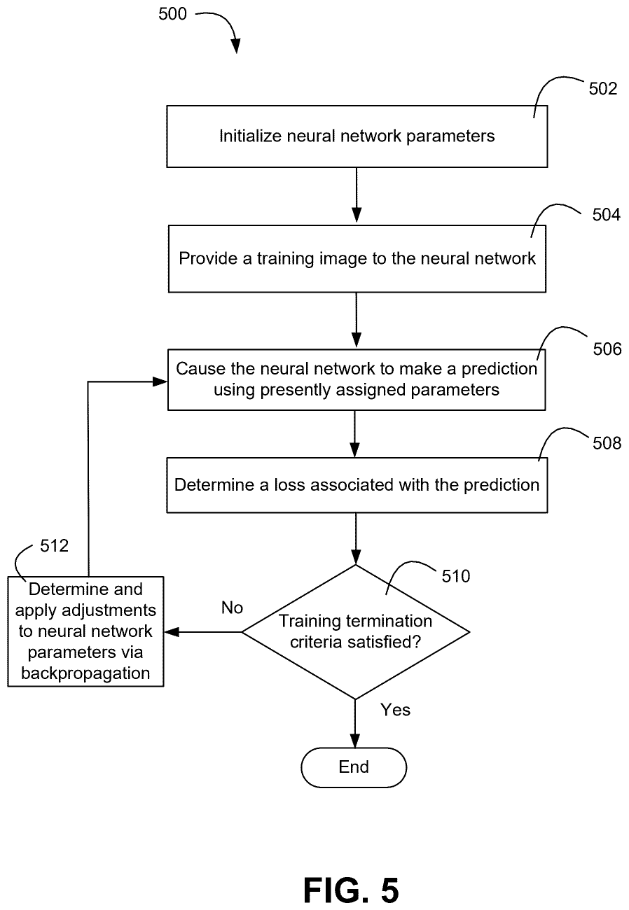

FIG. 5 illustrates example operations 500 that may be associated with training an artificial neural network (e.g., which may be configured to implement one or more of the ML models described herein) to perform one or more of the tasks described herein. As shown in FIG. 5, training operations 500 may include initializing the operating parameters of the neural network (e.g., weights associated with various layers of the neural network) at 502, for example, by sampling from a probability distribution or by copying the parameters of another neural network having a similar structure. The training operations may further include providing one or more first inputs (e.g., an image to be classified) to the neural network at 504 and causing the neural network to make a prediction at 506 (e.g., about a token, a caption, a sentence, etc.) using presently assigned network parameters. At 508, the training operations may further include determining a loss associated with the prediction, for example, based on a difference between the prediction and corresponding ground truth. The loss may be calculated using various types of loss functions, such as, e.g., a mean squared error (MSE) based loss function, an L1/L2 based loss function, a contrastive loss function, etc.

At 510, the training operations may further include determining whether one or more training termination criteria have been satisfied. For example, the training termination criteria may be determined to have been satisfied if the difference between the prediction and the ground truth falls below a predetermined threshold value. If the determination at 510 is that the training termination criteria are satisfied, the training may end. Otherwise, the presently assigned network parameters may be adjusted at 512, for example, by backpropagating a gradient descent of the loss through the network, before the training returns to 506.

For simplicity of explanation, the training operations are depicted and described herein with a specific order. It should be appreciated, however, that the training operations may occur in various orders, concurrently, and/or with other operations not presented or described herein. Furthermore, it should be noted that not all operations that may be included in the training process are depicted and described herein, and not all illustrated operations are required to be performed.

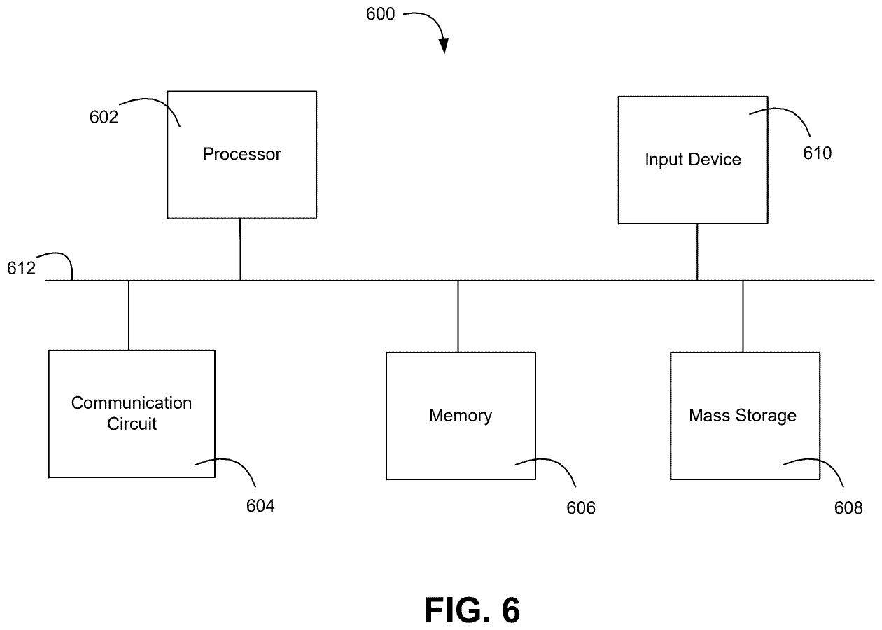

The systems, methods, and/or instrumentalities described herein may be implemented using one or more processors, one or more storage devices, and/or other suitable accessory devices such as display devices, communication devices, input/output devices, etc. FIG. 6 is a block diagram illustrating an example apparatus 600 that may be configured to perform the tasks described herein. As shown, apparatus 600 may include a processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked, or clustered) with other computation devices to perform the functions described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description. The scope of the disclosure should, therefore, be determined with reference to the appended claims, along with the full scope of equivalents to which such claims are entitled.

The term “computer-readable storage medium” used herein may include any tangible medium that is capable of storing or encoding a set of instructions for execution by a computer that cause the computer to perform any one or more of the methods described herein. The term “computer-readable storage medium” used herein may include, but not be limited to, solid-state memories, optical media, and magnetic media.

What is claimed is:

1. An apparatus, comprising:
one or more processors configured to:
obtain at least a first type of data associated with a medical procedure and a second type of data associated with the medical procedure;
generate, using a first machine learning (ML) model, first textual descriptions based on the first type of data, wherein the first textual descriptions are associated with multiple temporal levels;
generate, using a second ML model, second textual descriptions based on the second type of data, wherein the second textual descriptions are also associated with the multiple temporal levels;
produce a raw medical report that describes the medical procedure based at least on the first textual descriptions and the second textual descriptions, wherein the first textual descriptions and the second textual descriptions are aggregated in the raw medical report based on the multiple temporal levels with which the first textual descriptions and the second textual descriptions are associated; and
refine the raw medical report based on a large language model (LLM).

2. The apparatus of claim 1, wherein the first type of data includes a video recording of the medical procedure, and the first ML model includes a vision-language model configured to extract visual features from the video recording and generate the first textual descriptions based on the extracted visual features.

3. The apparatus of claim 2, wherein the second type of data includes an audio recording of the medical procedure, and the second ML model includes a speech recognition model configured to extract sound features from the audio recording and generate the second textual descriptions based on the extracted sound features.

4. The apparatus of claim 2, wherein the second type of data includes patient vital signs, patient medical records, or logs of a device used during the medical procedure, and wherein the second ML model includes an ML model configured to extract features from the patient vital signs, the patient medical records, or the logs of the device used during the medical procedure, the second ML model further configured to map the extracted features to the second textual descriptions.

5. The apparatus of claim 2, wherein the vision-language model is configured to determine, for each frame of the video recording, one or more region-wise tokens each indicative of a person or object detected in a corresponding region, and wherein, for each frame of the video recording, the vision-language model is further configured to determine a caption that describes the frame.

6. The apparatus of claim 1, wherein each of the multiple temporal levels corresponds to a respective time spot or step of the medical procedure.

7. The apparatus of claim 1, wherein the one or more processors being configured to produce the raw medical report comprises the one or more processors being configured to concatenate, for each temporal level of the multiple temporal levels, one or more of the first textual descriptions that correspond to the temporal level with one or more of the second textual descriptions that correspond to the temporal level.

8. The apparatus of claim 7, wherein the one or more processors being configured to produce the raw medical report further comprises the one or more processors being configured to aggregate, across the multiple temporal levels, the one or more of the first textual descriptions and the one or more of the second textual descriptions that are concatenated at each temporal level.

9. The apparatus of claim 1, wherein the LLM utilizes a transformer architecture and has over one billion parameters, the LLM configured to refine the raw medical report based on a predefined report structure or predefined report language.

10. The apparatus of claim 1, wherein the LLM is pre-trained to detect abnormalities in the raw medical report, and wherein the one or more processors being configured to refine the raw medical report based on the LLM comprises the one or more processors being configured to provide an indication of the abnormalities detected in the raw medical report.

11. The apparatus of claim 1, wherein the LLM is pre-trained to replace a medical terminology included in the raw medical report with descriptive texts, and wherein the one or more processors being configured to refine the raw medical report based on the LLM comprises the one or more processors being configured to replace the medical terminology with the descriptive texts.

12. The apparatus of claim 1, wherein the LLM is pre-trained to determine, based on the first type of data or the second type of data, standard operations associated with the medical procedure and actual operations being performed in the medical procedure, and wherein the one or more processors are further configured to detect inconsistency between the actual operations and the standard operations, and provide an indication of the inconsistency.

13. A method for automatic report generation, the method comprising:
obtaining at least a first type of data associated with a medical procedure and a second type of data associated with the medical procedure;
generating, using a first machine learning (ML) model, first textual descriptions based on the first type of data, wherein the first textual descriptions are associated with multiple temporal levels;
generating, using a second ML model, second textual descriptions based on the second type of data, wherein the second textual descriptions are also associated with the multiple temporal levels;
producing a raw medical report that describes the medical procedure based at least on the first textual descriptions and the second textual descriptions, wherein the first textual descriptions and the second textual descriptions are aggregated in the raw medical report based on the multiple temporal levels with which the first textual descriptions and the second textual descriptions are associated; and
refining the raw medical report based on a large language model (LLM).

14. The method of claim 13, wherein the first type of data includes a video recording of the medical procedure, and the first ML model includes a vision-language model configured to extract visual features from the video recording and generate the first textual descriptions based on the extracted visual features.

15. The method of claim 14, wherein the second type of data includes an audio recording of the medical procedure, patient vital signs, patient medical records, or logs of a device used during the medical procedure, and wherein the second ML model includes an ML model configured to extract features from the audio recording, the patient vital signs, the patient medical records, or the logs of the device used during the medical procedure, the second ML model further configured to map the extracted features to the second textual descriptions.

16. The method of claim 14, wherein the vision-language model is configured to determine, for each frame of the video recording, one or more region-wise tokens each indicative of a person or object detected in a corresponding region, and wherein, for each frame of the video recording, the vision-language model is further configured to determine a caption that describes the frame.

17. The method of claim 13, wherein each of the multiple temporal levels corresponds to a respective time spot or step of the medical procedure.

18. The method of claim 13, wherein producing the raw medical report comprises concatenating, for each temporal level of the multiple temporal levels, one or more of the first textual descriptions that correspond to the temporal level with one or more of the second textual descriptions that correspond to the temporal level.

19. The method of claim 18, wherein producing the raw medical report further comprises aggregating, across the multiple temporal levels, the one or more of the first textual descriptions and the one or more of the second textual descriptions that are concatenated at each temporal level.

20. The method of claim 13, wherein the LLM is configured to refine the raw medical report based on a predefined report structure or predefined report language.
